(19) United States
(12) Patent Application Publication
(10) Pub. No.: US 2026/0021056 A1
(43) Pub. Date: Jan. 22, 2026

(54) SKIN PATCH SHEET

(71) Applicant: TOPPAN HOLDINGS INC., Tokyo (JP)

(72) Inventors: Saeko NOMURA, Tokyo (JP); Natsumi MIYAUCHI, Tokyo (JP); Junya TANABE, Tokyo (JP)

(73) Assignee: TOPPAN HOLDINGS INC., Tokyo (JP)

(21) Appl. No.: 19/341,499

(22) Filed: Sep. 26, 2025

Related U.S. Application Data
(63) Continuation of application No. PCT/JP2024/005586, filed on Feb. 16, 2024.

(30) Foreign Application Priority Data
Mar. 31, 2023 (JP) 2023.057962

(51) Int. Cl. A61K 9/70; A61K 8/02; A61Q 19/00

(52) U.S. Cl. CPC A61K 9/7069; A61K 8/0208; A61Q 19/00; A61K 2800/87

(57) ABSTRACT

A patch tape includes a substrate layer in contact with a carrier sheet, an adhesive layer containing an active ingredient for skin and an adhesive, and a barrier layer located between the substrate layer and the adhesive layer and having a thickness of less than 5 μm. The patch tape is attached to the skin after the release sheet is peeled from the adhesive layer. The 180° peeling strength according to JIS Z 0237:2009 between the carrier sheet and the substrate layer is 550 mN/25 mm or less. In the patch tape, the tensile elongation at break according to JIS K 7161-1:2014 is 130% or more.

### CROSS-REFERENCE TO RELATED PATENT APPLICATIONS

The present application is a Bypass Continuation of International Patent Application No. PCT/JP2024/005586, filed Feb. 16, 2024, which claims priority to and the benefit of Japanese Patent Application No. 2023-057962, filed on Mar. 31, 2023. The contents of these applications are hereby incorporated by reference herein in their entireties.

### TECHNICAL FIELD

The present disclosure relates to a skin patch sheet.

### BACKGROUND

A first example of a patch sheet includes a flexible film, a drug non-adsorptive layer, an adhesive layer, and a release liner. The adhesive layer contains an acrylic adhesive and active ingredients. The active ingredients are crotamiton, oleic acid, and at least one of estradiol and a derivative thereof. In the patch sheet, the drug non-adsorptive layer is located between the film and the adhesive layer, thereby preventing the active ingredients from being absorbed by a support composed of the film and the drug non-adsorptive layer (see, for example, PTL 1).

A second example of a patch sheet includes a support film, a barrier layer, and an adhesive layer. The support film contains a urethane resin. The barrier layer contains an ethylene-vinyl alcohol copolymer. The adhesive layer contains an oil-based additive. In the patch sheet, the barrier layer is located between the support film and the adhesive layer, thus preventing the oil-based additive from migrating to the support film, and consequently maintaining the content of the oil-based additive in the adhesive layer (see, for example, PTL 2).

[Citation List] [Patent Literature] [0004] PTL 1: JP 2004-075537 A PTL 2: JP 2016-013993 A

### SUMMARY OF THE INVENTION

### Technical Problem

The substrate layer to be attached to the skin is required to have high tensile elongation at break, similar to that of polyurethane resin, with the aim to achieve conformability to the skin. On the other hand, such a substrate layer also has high adhesion to a carrier sheet made of a polyolefin resin or a polyester resin, making it difficult to peel the carrier sheet from the skin once a laminate of the patch tape and the carrier sheet has been attached.

Further, the barrier layer has higher rigidity than the substrate layer. Therefore, a difference in rigidity occurs between the barrier layer and the substrate layer. As a result, the patch tape may deform when it is stretched or compressed, or the patch tape may tear when it is peeled from the skin.

### Solution to Problem

An aspect of a skin patch tape includes a carrier sheet, a release sheet, and a patch tape located between the carrier sheet and the release sheet. The patch tape includes a substrate layer in contact with the carrier sheet, an adhesive layer containing an active ingredient for skin and an adhesive, and a barrier layer located between the substrate layer and the adhesive layer and having a thickness of less than 5 μm. The patch tape is attached to the skin after the release sheet is peeled from the adhesive layer. The 180° peeling strength according to JIS Z 0237:2009 between the carrier sheet and the substrate layer is 550 mN/25 mm or less. In the patch tape, the tensile elongation at break according to JIS K 7161-1:2014 is 130% or more.

### BRIEF DESCRIPTION OF THE DRAWINGS

FIG. 1 is a cross-sectional view showing a skin patch sheet.

FIG. 2 is a table showing the results of evaluating the physical property values of skin patch sheets.

FIG. 3 is a graph showing the relationship between displacement and test force in the skin patch sheet of Comparative Example 1.

FIG. 4 is a graph showing the relationship between displacement and test force in the skin patch sheet of Example 1.

FIG. 5 is a graph showing the relationship between displacement and test force in the skin patch sheet of Example 2.

FIG. 6 is a graph showing the relationship between displacement and test force in the skin patch sheet of Example 3.

FIG. 7 is a graph showing the relationship between displacement and test force in the skin patch sheet of Comparative Example 2.

FIG. 8 is a graph showing the relationship between displacement and test force in the skin patch sheet of Example 4.

FIG. 9 is a graph showing the relationship between displacement and test force in the skin patch sheet of Example 5.

FIG. 10 is a graph showing the relationship between displacement and test force in the skin patch sheet of Example 6.

FIG. 11 is a table showing the results of evaluating the handling properties of skin patch sheets.

### DETAILED DESCRIPTION

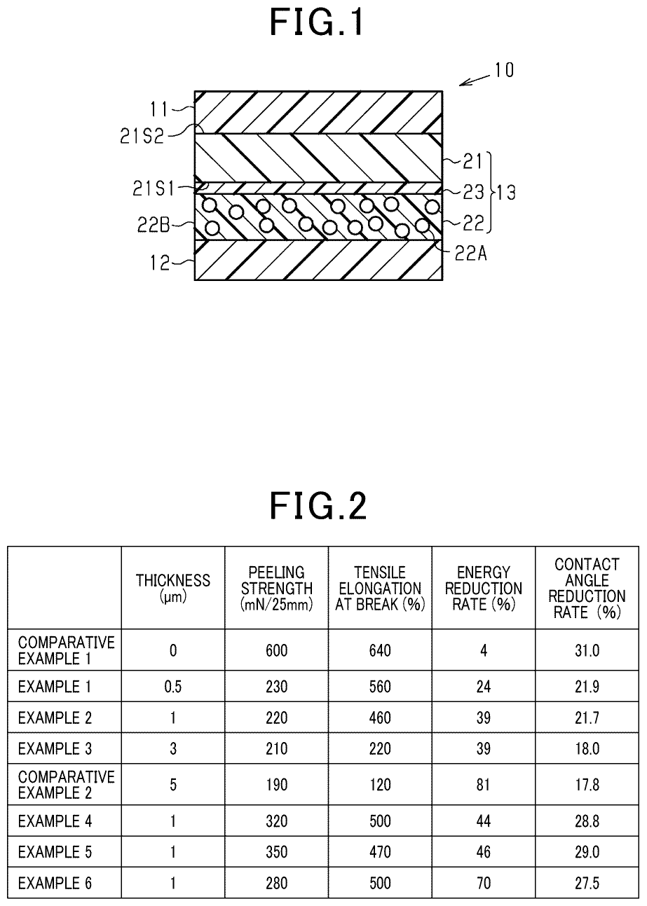

An embodiment of a skin patch sheet will be described with reference to FIGS. 1 to 11.

### Skin Patch Sheet

As shown in FIG. 1, a skin patch sheet (hereinafter also referred to as the patch sheet) 10 includes a carrier sheet 11, a release sheet 12, and a patch tape 13 located between the carrier sheet 11 and the release sheet 12. The patch tape 13 includes a substrate layer 21, an adhesive layer 22, and a barrier layer 23. The substrate layer 21 is in contact with the carrier sheet 11. The adhesive layer 22 contains an active ingredient 22A for skin and an adhesive 22B. The barrier layer 23 is located between the substrate layer 21 and the adhesive layer 22, and has a thickness of less than 5 μm. The patch tape 13 is attached to the skin after the release sheet 12 is peeled from the adhesive layer 22. At this time, the adhesive layer 22 of the patch tape 13 is in contact with the skin.

The patch sheet 10 satisfies the following conditions 1 and 2.

(Condition 1) The 180° peeling strength according to JIS Z 0237:2009 between the carrier sheet 11 and the substrate layer 21 is 550 mN/25 mm or less.

(Condition 2) In the patch tape 13, the tensile elongation at break according to JIS K 7161-1:2014 is 130% or more.

The patch sheet 10 of the present disclosure, in which the 180° peeling strength between the carrier sheet 11 and the substrate layer 21 is 550 mN/25 mm or less, facilitates the peeling of the carrier sheet 11 from the substrate layer 21. Further, the thickness of the barrier layer 23 being less than 5 μm prevents the deformation of the patch tape 13 when the patch tape 13 is stretched or compressed, and also prevents tearing of the patch tape 13 when the patch tape 13 is peeled from the skin.

The 180° peeling strength between the carrier sheet 11 and the substrate layer 21 is a value measured by a method according to JIS Z 0237:2009 “Testing methods of pressure-sensitive adhesive tapes and sheets.” The patch sheet 10 is configured so that the 180° peeling strength between the carrier sheet 11 and the substrate layer 21 is higher than the 180° peeling strength between the release sheet 12 and the adhesive layer 22. The patch sheet 10 is configured so that the 180° peeling strength between the layers included in the patch tape 13 is higher than the 180° peeling strength between the carrier sheet 11 and the substrate layer 21.

The tensile elongation at break of the patch tape 13 can be determined according to JIS K 7161-1:2014 (ISO 527-1) “Plastics—Determination of tensile properties—Part 1: General principles” and JIS K 7127:1999 (ISO 527-3) “Plastics—Determination of tensile properties—Part 3: Test conditions for films and sheets.” The tensile elongation at break can be calculated by using a tensile strain at break when an object to be measured does not have a yield point, and a nominal tensile strain at break when an object to be measured has a yield point.

In the patch tape 13 provided in the patch sheet 10 of the present embodiment, the adhesive layer 22 is in contact with the release sheet 12, and the substrate layer 21 and the adhesive layer 22 are in contact with the barrier layer 23. The substrate layer 21 includes a first surface 21S1 in contact with the barrier layer 23, and a second surface 21S2 on an opposite side to the first surface 21S1.

The patch sheet 10 preferably satisfies at least one of the following conditions 3 and 4. That is, the patch sheet 10 may satisfy at least one of the conditions 3 and 4, or may satisfy both of the conditions 3 and 4.

(Condition 3) When the patch tape 13 is subjected to 100% elongation according to JIS K 7161-1:2014 twice in a row, the decrease rate of energy at the second 100% elongation is 70% or less, with energy at the first 100% elongation as a reference value. Each energy is an integral value of test force and displacement obtained by the 100% elongation.

(Condition 4) The active ingredient 22A is hydrophilic, and regarding a contact angle according to the sessile drop method of JIS R 3257:1999, the decrease rate of the contact angle 20 seconds after a drop of water is dropped is 25% or less, with the contact angle when the drop of water is dropped on the second surface 21S2 as a reference value.

The 100% elongation of the patch tape 13 is performed by a method according to JIS K 7161-1:2014. At this time, a graph showing the relationship between the test force applied to the patch tape 13 and displacement, which is the amount of stretching of the patch tape 13, can be obtained. The 100% elongation of an object refers to the state in which the object has been stretched to 2×L1, where L1 is the initial length before stretching. The energy at 100% elongation refers to a cumulative value of the test force applied to the object until it reaches 100% elongation and the displacement of the object. That is, in a graph showing the relationship between test force and displacement, with the test force set on the vertical axis and the displacement set on the horizontal axis, the energy at 100% elongation is the area enclosed by the graph showing the relationship between test force and displacement, the horizontal axis, and a line segment parallel to the vertical axis and passing through a length corresponding to 100% elongation on the horizontal axis. The decrease rate of the energy at the second 100% elongation is calculated by the following formula, with the energy at the first 100% elongation as the reference value.

$$Energy decrease rate = \{ ( E 1 - E 2 ) / E 1 \} \times 100$$

In the above formula, E1 is the energy at the first 100% elongation. E2 is the energy at the second 100% elongation.

The contact angle on the second surface 21S2 of the substrate layer 21 is a value measured by a method according to JIS R 3257:1999 “Testing method of wettability of glass substrate.” The decrease rate of the contact angle 20 seconds after a drop of water is dropped is calculated by the following formula, with the contact angle when the drop of water is dropped on the second surface 21S2 as the reference value.

$$Contact angle decrease rate = \{ ( C 1 - C 2 ) / C 1 \} \times 100$$

In the above formula, C1 is the contact angle when a drop of water is dropped on the second surface 21S2. C2 is the contact angle 20 seconds after the drop of water is dropped on the second surface 21S2.

The rigidity of the barrier layer 23 is higher than the rigidity of the substrate layer 21. In other words, the tensile elongation at break of the substrate layer 21 is higher than the tensile elongation at break of the barrier layer 23. Since the substrate layer 21 has high tensile elongation at break, the substrate layer 21 is stretched while the force stretching the substrate layer 21 is applied thereto, whereas it can return to its pre-stretched state when the stretching force is released. That is, the substrate layer 21 has high elasticity. In contrast, the barrier layer 23 with lower tensile elongation at break is less likely to return to its pre-stretched state even when the force stretching the barrier layer 23 is released. That is, the elasticity of the barrier layer 23 is less than the elasticity of the substrate layer 21. Thus, since the elasticity of the substrate layer 21 and the elasticity of the barrier layer 23 are different from each other, when the force stretching the patch tape 13 is released, the patch tape 13 is curled at the edges, and the barrier layer 23 is cracked.

In this respect, according to the patch sheet 10 that satisfies the condition 3, the decrease rate of the energy at the second 100% elongation is 70% or less, with the energy at the first 100% elongation as the reference value. Therefore, even the patch sheet 10, which includes the barrier layer 23, can maintain elasticity. This can prevent curling of the patch tape 13 at the edges and cracking of the barrier layer 23 when the force stretching the patch tape 13 is released.

The hydrophilic active ingredient 22A migrates within the substrate layer 21 along the direction from the adhesive layer 22 toward the carrier sheet 11. When the substrate layer 21 contains a polyurethane resin, and the surface of the carrier sheet 11 in contact with the substrate layer 21 contains a polyolefin resin, the active ingredient 22A remains between the substrate layer 21 and the carrier sheet 11. Therefore, it is possible to use the contact angle of the second surface 21S2, which exhibits hydrophilicity in the second surface 21S2 of the substrate layer 21, as an index indicating the degree of migration of the active ingredient 22A.

According to the patch sheet 10 that satisfies the condition 4, the barrier layer 23 prevents the active ingredient 22A from migrating from the adhesive layer 22 towards the substrate layer 21, thereby preventing increase in the adhesion between the carrier sheet 11 and the substrate layer 21.

The surface of the carrier sheet 11 in contact with the substrate layer 21 may be made of a polyolefin resin. This can increase the effectiveness of facilitating the peeling of the carrier sheet 11 from the substrate layer 21. The polyolefin resin may be, for example, a polypropylene resin or a polyethylene resin. The carrier sheet 11 may be a resin film, or paper laminated with a resin film. The resin film may be a stretched film or a non-stretched film. The surface of the carrier sheet 11 in contact with the substrate layer 21 may be processed to facilitate the peeling of the substrate layer 21 from the carrier sheet 11. The processing on the surface in contact with the substrate layer 21 may be, for example, embossing. The carrier sheet 11 may have a thickness of, for example, 30 μm or more and 400 μm or less.

The release sheet 12 is configured so that the peeling strength between the release sheet 12 and the adhesive layer 22 is less than the peeling strength between the carrier sheet 11 and the substrate layer 21. The release sheet 12 is made of a synthetic resin. The release sheet 12 is formed from, for example, a substrate sheet and a release layer. The release layer is laminated on the substrate sheet. When the release sheet 12 includes a substrate sheet and a release layer, the release layer is in contact with the adhesive layer 22 of the patch tape 13. The substrate sheet may be made of, for example, polyethylene terephthalate (PET). The substrate sheet may be any of a uniaxially stretched sheet, a biaxially stretched sheet, and a non-stretched sheet. The release layer may be made of, for example, a silicone resin.

The release sheet 12 may consist only of the substrate sheet described above. In this case, the surface of the substrate sheet in contact with the adhesive layer 22 may be processed to facilitate the peeling of the adhesive layer 22 from the substrate sheet. The processing on the contact surface of the substrate sheet may be, for example, embossing. The release sheet 12 may have a thickness of, for example, 12 μm or more and 350 μm or less.

The substrate layer 21 is made of a synthetic resin. The synthetic resin for forming the substrate layer 21 may be, for example, a polyurethane resin. Accordingly, a substrate layer 21 having high adhesion suitability can be provided. The substrate layer 21 may have a thickness of, for example, 5 μm or more and 30 μm or less. The substrate layer 21 made of a polyurethane resin and having a small thickness is easily stretched even when a small amount of external force is applied to the substrate layer 21 to stretch the substrate layer 21. Therefore, the substrate layer 21 has high conformability to the shape of the skin to which the patch tape 13 is attached, and can have high adhesion to the skin.

On the other hand, the substrate layer 21, which has high conformability to the shape of the skin, also has high conformability to the carrier sheet 11, which covers the substrate layer 21. Therefore, when the force to peel the carrier sheet 11 from the substrate layer 21 acts on the carrier sheet 11, the substrate layer 21 easily deforms according to the deformation of the carrier sheet 11.

The substrate layer 21 may be made of a synthetic resin other than polyurethane resins. Examples of the synthetic resin other than polyurethane resins include polyvinylidene fluoride resin, ethylene-vinyl acetate copolymer resin, polypropylene resin, and polyethylene terephthalate resin.

The adhesive 22B contained in the adhesive layer 22 is made of a synthetic resin. The synthetic resin for forming the adhesive 22B may be, for example, a polyurethane resin. The adhesive layer 22 may have a thickness of, for example, 5 μm or more and 25 μm or less.

The active ingredient 22A may be, for example, a cosmetic component, a beauty component, a pharmaceutical component, or the like. The active ingredient 22A may be a hydrophilic compound. The hydrophilic compound may be, for example, a polyhydric alcohol. The polyhydric alcohol may be, for example, at least one of glycerin, propylene glycol, and polyethylene glycol. That is, the adhesive layer 22 may contain only one or two or more of such hydrophilic compounds.

The barrier layer 23 is made of a synthetic resin. This can prevent increase in the rigidity of the barrier layer 23. The barrier layer 23 may be made of, for example, any of a polyvinyl butyral resin, a polyester resin, and a polyurethane resin. When the substrate layer 21 and the barrier layer 23 are both made of a polyurethane resin, the hard segment content of the barrier layer 23 is higher than the hard segment content of the substrate layer 21. The barrier layer 23 is preferably made of a polyvinyl butyral resin. Because the barrier layer 23 is made of a polyvinyl butyral resin, it is possible to prevent the migration of the active ingredient while maintaining flexibility. The barrier layer 23 may have a thickness of, for example, 0.1 μm or more and less than 5 μm. The barrier layer 23 may have a single-layer structure or may have a multilayer structure.

### EXAMPLES

Examples and Comparative Examples will be described with reference to FIGS. 2 to 11.

### Comparative Example 1

A biaxially oriented polypropylene (OPP) film (manufactured by Futamura Chemical Co., Ltd., FOR-MP, thickness: 40 μm) having a matte surface as one of a pair of opposing surfaces was prepared as a carrier sheet. A first aqueous polyurethane containing ether-based polyol (manufactured by Mitsui Chemicals, Inc., TAKELAC WS-6021) and a second aqueous polyurethane containing ether-based polyol (manufactured by Mitsui Chemicals, Inc., W-6020) were mixed to prepare a coating liquid. At this time, the weight ratio of the first aqueous polyurethane to the second aqueous polyurethane was set as follows.

First aqueous polyurethane:second aqueous polyurethane=10:1

Then, the coating liquid was applied to the matte surface of the OPP film, after which the coating liquid was dried to form a precursor layer. Subsequently, the precursor layer was left at room temperature for aging to thereby obtain a substrate layer having a thickness of 15 μm.

A release film composed of a PET film and a release layer made of a silicone resin (Toray Industries, Inc., Cerapeel WZ, thickness: 75 μm) (Cerapeel is a registered trademark) was prepared as a release sheet. Next, a urethane-based adhesive (manufactured by Toyochem Co., Ltd., SP-205) as a base resin, and a curing agent (manufactured by Toyochem Co., Ltd., T-501B) were prepared. Further, concentrated glycerin (manufactured by Kanto Chemical Co., Inc., 17029-08, in accordance with the Japanese Standards of Quasi-Drug Ingredients) was prepared as an active ingredient for skin. After the curing agent and concentrated glycerin were added to the base resin, the base resin, curing agent, and concentrated glycerin were stirred to obtain a coating liquid for forming an adhesive layer. At this time, the weight of glycerin was set to 40% relative to the solid content of the base resin. The coating liquid was applied to the release film, and the coating liquid was then dried to obtain an adhesive layer having a thickness of 15 μm.

Then, the substrate and the adhesive layer were bonded together, thereby obtaining a laminate in which the OPP film, a patch tape, and the release film were laminated in this order. In this way, a patch body of Comparative Example 1 was obtained.

### Example 1

In the patch sheet of Comparative Example 1, after an aqueous polyurethane precursor layer was formed, a coating liquid in which a polyvinyl butyral resin was dissolved in ethanol (manufactured by Dainichiseika Color & Chemicals Mfg. Co., Ltd.) was applied to form a coating film, after which the coating film was dried, thereby forming a barrier layer having a thickness of 0.5 μm. A patch sheet of Example 1 was obtained in the same manner as for the patch sheet of Comparative Example 1 except for the above points. When the adhesive layer was attached to the laminate of the substrate layer and the barrier layer, the adhesive layer was attached to the laminate so that the barrier layer was sandwiched between the substrate layer and the adhesive layer.

### Example 2

A patch sheet of Example 2 was obtained in the same manner as in Example 1, except that the thickness of the barrier layer in the patch sheet of Example 1 was changed to 1 μm.

### Example 3

A patch sheet of Example 3 was obtained in the same manner as in Example 1, except that the thickness of the barrier layer in the patch sheet of Example 1 was changed to 3 μm.

### Comparative Example 2

A patch sheet of Comparative Example 2 was obtained in the same manner as in Example 1, except that the thickness of the barrier layer in the patch sheet of Example 1 was changed to 5 μm.

### Example 4

In the patch sheet of Example 2, in place of the coating liquid in which a polyvinyl butyral resin was dissolved in ethanol, a water-dispersed polyester resin (manufactured by Toyobo Co., Ltd., Vylonal MD-1480) was applied to form a coating film, after which the coating film was dried, thereby forming a barrier layer having a thickness of 1 μm. A patch sheet of Example 4 was obtained in the same manner as in Example 2 except for the above points.

### Example 5

In the patch sheet of Example 2, in place of the coating liquid in which a polyvinyl butyral resin was dissolved in ethanol, a polyolefin aqueous dispersion (manufactured by Mitsui Chemicals, Inc., Chemipearl S500) was applied to form a coating film, after which the coating film was dried, thereby forming a barrier layer having a thickness of 1 μm. A patch sheet of Example 5 was obtained in the same manner as in Example 2 except for the above points.

### Example 6

In the patch sheet of Example 2, in place of the coating liquid in which a polyvinyl butyral resin was dissolved in ethanol, a polyurethane aqueous dispersion (manufactured by Mitsui Chemicals, Inc., WPB341) was applied to form a coating film, after which the coating film was dried, thereby forming a barrier layer having a thickness of 1 μm. A patch sheet of Example 6 was obtained in the same manner as in Example 2 except for the above points.

### Evaluation Method

### [180° Peeling Strength Between Carrier Sheet and Substrate Layer]

The peeling strength of the substrate layer relative to the carrier sheet was measured by a method according to JIS Z 0237:2009 “Testing methods of pressure-sensitive adhesive tapes and sheets.” In the measurement of the peeling strength, a universal tester (manufactured by Shimadzu Corporation, Autograph AGS-X, load cell 5 kN) was used. When measuring the peeling strength, a test piece having a width of 25 mm was cut out. Then, the substrate layer was fixed to the universal tester, and the strength when peeling the carrier sheet at 180° with respect to the substrate was calculated.

### Tensile Elongation at Break of Patch Tape

The patch tape of each patch sheet was cut into a dumbbell shape (test piece type 5) by a method according to JIS K 7127:1999 “Plastics—Determination of tensile properties—Part 3: Test conditions for films and sheets,” thereby obtaining test pieces. The tensile elongation at break of each test piece was measured by a method according to JIS K 7161-1:2014. Specifically, the tensile elongation at break was measured by using a universal tester (manufactured by Shimadzu Corporation, Autograph AGS-X, load cell 5 kN). At this time, the tensile speed was set to 300 mm/min, and the gauge length was set to 25 mm. The test piece was pulled to determine the elongation at break of the test piece.

### Energy Decrease Rate of Patch Tape

A test piece was made from each patch sheet in the same manner as when measuring the tensile elongation at break of the patch tape. Further, the test piece was pulled until the displacement of the test piece reached 25 mm under the same conditions as when measuring the tensile elongation at break of the patch tape. In this way, the first 100% elongation was carried out. Thereafter, the test force acting on the test piece was released, and subsequently the second 100% elongation was carried out.

Then, based on a graph obtained from each test piece, the integral value of the test force applied to the test piece until the displacement, i.e., the amount of stretching of the test piece, reached 25 mm, and the displacement was calculated. In other words, the area enclosed by the graph showing the relationship between test force and displacement, the horizontal axis, and a line segment parallel to the vertical axis and passing through 25 mm on the horizontal axis was calculated. In this way, the energy at the first 100% elongation and the energy at the second 100% elongation were calculated for each test piece. Then, the energy decrease rate was calculated using the following formula.

$$Energy decrease rate = \{ ( E 1 - E 2 ) / E 1 \} \times 100$$

In the above formula, E1 is the energy at the first 100% elongation, and E2 is the energy at the second 100% elongation.

### Contact Angle Decrease Rate of Substrate Layer

For each patch sheet, the contact angle of pure water on the second surface of the substrate layer from which the carrier sheet was peeled was measured. At this time, the contact angle of pure water on each patch sheet was measured by a method according to the sessile drop method of JIS R 3257:1999 “Testing method of wettability of glass substrate” using a contact angle meter (manufactured by Kyowa Interface Science Co., Ltd., PCA-1). At this time, the contact angles were measured at five different locations on each patch sheet, and the average value of the contact angles measured at the five locations was set as the contact angle for each patch sheet. In addition, the change in contact angle over time was measured from the time a drop of water was dropped on the second surface of the substrate layer to 20 seconds later. Then, the decrease rate of the contact angle 20 seconds after a droplet of water was dropped was calculated by the following formula, with the contact angle when the drop of water was dropped on the second surface of the substrate layer as the reference value.

$$Contact angle decrease rate = \{ ( C 1 - C 2 ) / C 1 \} \times 100$$

In the above formula, C1 is the contact angle when a drop of water is dropped on the second surface. C2 is the contact angle 20 seconds after the drop of water is dropped on the second surface. The contact angle decrease rate was evaluated in the following three stages.

Less than 25%: A (excellent): The migration of glycerin was more prevented.

25% or more and less than 30%: B (good): The migration of glycerin was prevented.

30% or more: C (poor): The migration of glycerin was not prevented.

### Handling Properties

### [Peeling of Carrier Sheet]

In each Example and each Comparative Example, the shape of the patch sheet was set to a square with each side measuring 10 cm. After the release sheet was peeled from the patch sheet, the adhesive layer was attached to the skin of a subject. Next, one corner portion of the carrier sheet was peeled to form a starting point for peeling the carrier sheet from the substrate layer, after which the carrier sheet was peeled from the substrate layer. At this time, how the carrier sheet was peeled was evaluated in the following two stages.
A: No resistance was felt when the carrier sheet was peeled from the substrate layer.
C: Resistance was felt when the carrier sheet was peeled from the substrate layer.

### [Elasticity of Patch Tape]

A test piece with a length of 50 mm and a width of 10 mm was cut from each patch sheet. Subsequently, the release sheet was peeled from each test piece, and the adhesive layer of the patch tape was then attached to the skin of a subject while stretching the patch tape. At this time, the elasticity of the patch tape was evaluated in the following two stages.
A: The patch tape could be attached to the skin without wrinkles, unaffected by any deformation of the patch tape.
C: Due to the deformation of the patch tape, wrinkles formed on the patch tape attached to the skin.

### [Tearing of Patch Tape]

A test piece with a length of 50 mm and a width of 10 mm was cut from each patch sheet. After the release sheet was peeled from each test piece, the adhesive layer was attached to the skin of a subject. Subsequently, after the carrier sheet was peeled, the patch tape was peeled from the skin of the subject. At this time, how the patch tape was peeled was evaluated in the following two stages.
A: The patch tape could be peeled off in a single motion without being torn while peeling it from the skin.
C: The patch tape tore at least once while peeling it from the skin.

### [Evaluation Results]

The evaluation results of the 180° peeling strength, tensile elongation at break, energy decrease rate, and contact angle decrease rate were each as shown in FIG. 2.

As shown in FIG. 2, it was recognized that the 180° peeling strength was 230 mN/25 mm in Example 1, 220 mN/25 mm in Example 2, and 210 mN/25 mm in Example 3. It was also recognized that the 180° peeling strength was 320 mN/25 mm in Example 4, 350 mN/25 mm in Example 5, and 280 mN/25 mm in Example 6. It was also recognized that the 180° peeling strength was 600 mN/25 mm in Comparative Example 1, and 190 mN/25 mm in Comparative Example 2.

It was recognized that the tensile elongation at break was 560% in Example 1, 460% in Example 2, and 220% in Example 3. It was also recognized that the tensile elongation at break was 500% in Example 4, 470% in Example 5, and 500% in Example 6. It was also recognized that the tensile elongation at break was 640% in Comparative Example 1, and 120% in Comparative Example 2.

It was recognized that the energy decrease rate was 24% in Example 1, 39% in Example 2, and 39% in Example 3. It was also recognized that the energy decrease rate was 44% in Example 4, 46% in Example 5, and 70% in Example 6. It was recognized that the energy decrease rate was 4% in Comparative Example 1, and 81% in Comparative Example 2.

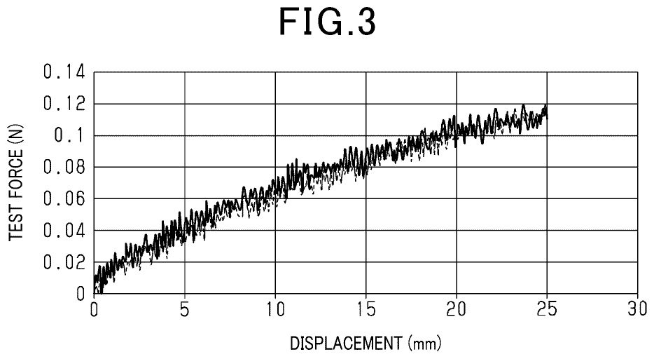

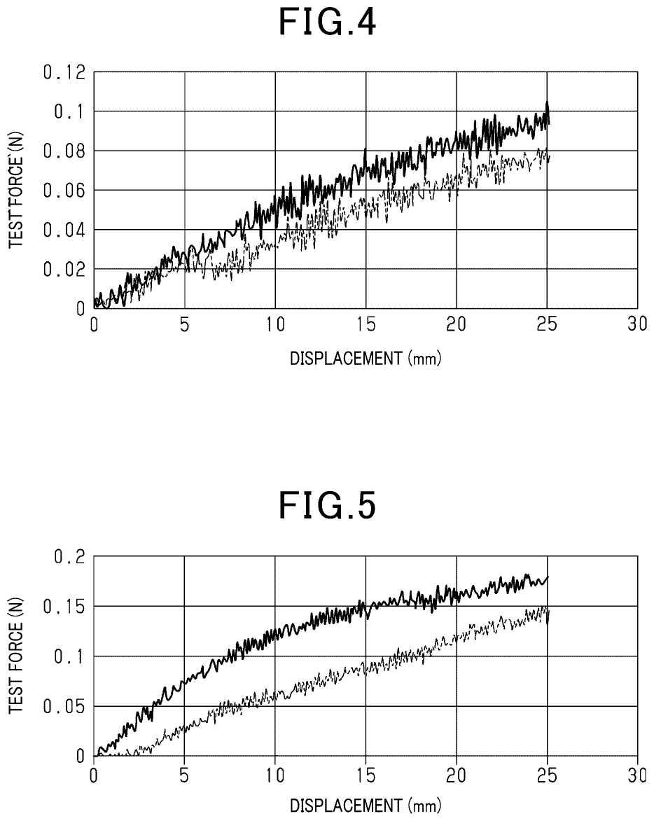

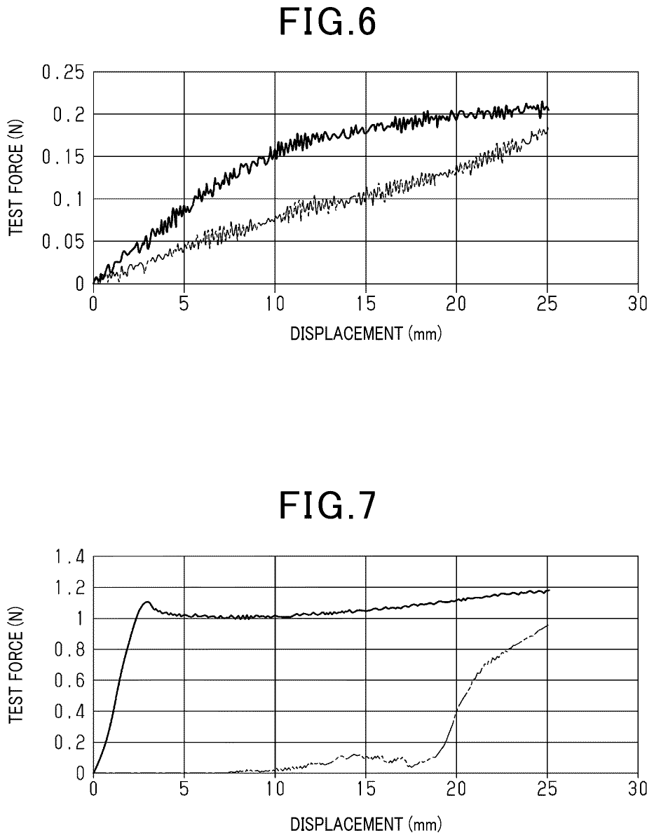

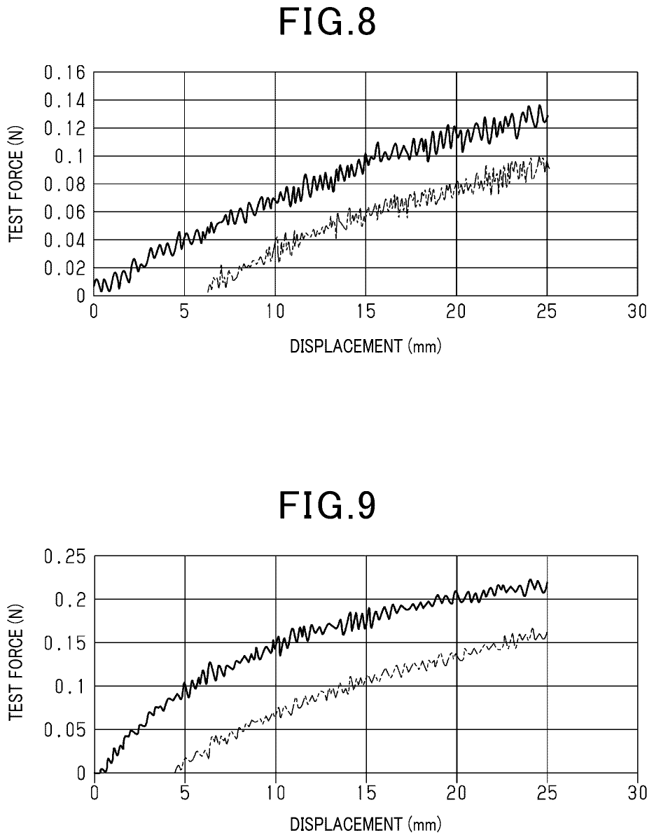

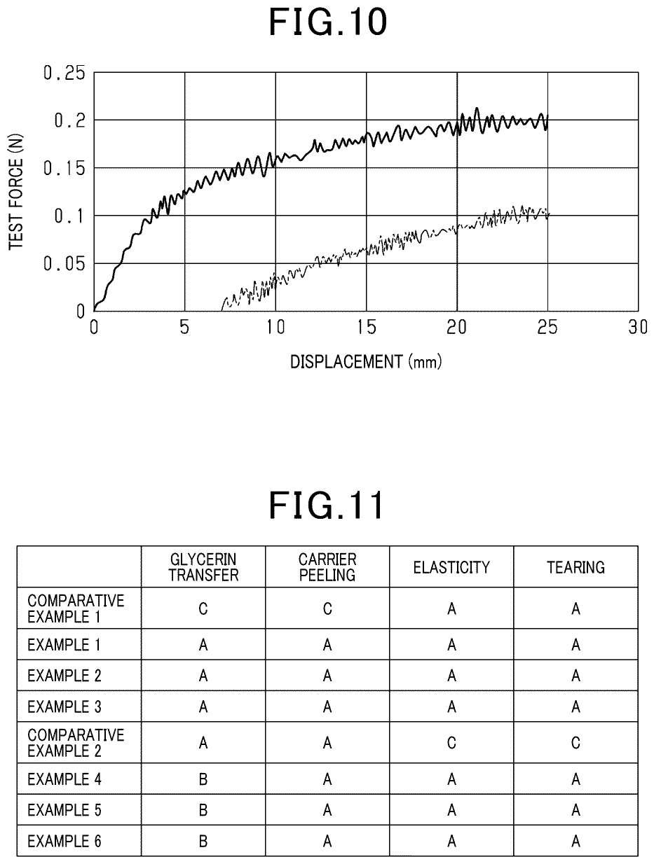

FIGS. 3 to 10 show graphs each showing the relationship between test force and displacement obtained when each patch tape was subjected to 100% elongation. FIG. 3 is a graph obtained for the patch sheet of Comparative Example 1, FIG. 4 is a graph obtained for the patch sheet of Example 1, and FIG. 5 is a graph obtained for the patch sheet of Example 2. FIG. 6 is a graph obtained for the patch sheet of Example 3, FIG. 7 is a graph obtained for the patch sheet of Comparative Example 2, and FIG. 8 is a graph obtained for the patch sheet of Example 4. FIG. 9 is a graph obtained for the patch sheet of Example 5, and FIG. 10 is a graph obtained for the patch sheet of Example 6. In FIGS. 3 to 10, the graphs obtained from the first 100% elongation are shown as solid lines, while the graphs obtained from the second 100% elongation are shown as dashed lines.

It was recognized that the contact angle decrease rate was 21.9% in Example 1, 21.7% in Example 2, and 18.0% in Example 3. It was recognized that the contact angle decrease rate was 28.8% in Example 4, 29.0% in Example 5, and 27.5% in Example 6. It was recognized that the contact angle decrease rate was 31.0% in Comparative Example 1, and 17.8% in Comparative Example 2.

As shown in FIG. 11, it was recognized in Examples 1 to 6 that the evaluation results of the peeling of the carrier sheet, the elasticity of the patch tape, and the tearing of the patch tape were all “A.” In contrast, it was recognized that in Comparative Example 1, which did not have a barrier layer, the evaluation results of the peeling of the carrier sheet were “C,” and that in Comparative Example 2, which had a thick barrier layer, the evaluation results of the elasticity of the patch tape and the tearing of the patch tape were each “C.”

These results suggest that because the 180° peeling strength between the carrier sheet and the substrate layer is 550 mN/25 mm or less, it is possible to facilitate the peeling of the carrier sheet from the substrate layer. Further, it can be said that from the viewpoint of facilitating the peeling of the carrier sheet from the substrate layer, the 180° peeling strength is preferably 350 mN/25 mm or less, and more preferably 230 mN/25 mm or less.

It can also be said that because the tensile elongation at break of the patch tape is 130% or more, it is possible to enhance conformability to the skin. It can be said that from the viewpoint of enhancing conformability to the skin, the tensile elongation at break is preferably 220% or more, and more preferably 460% or more.

It can also be said that because the thickness of the barrier layer is less than 5 μm, the elasticity of the patch tape is maintained at a high level, and the tearing of the patch tape is prevented. It can be said that from the viewpoint of maintaining the elasticity of the patch tape at a high level, the thickness of the barrier layer is preferably 3 μm or less, and more preferably 1 μm or less.

It can be said that because the contact angle decrease rate in Examples 1 to 6 is lower than that in Comparative Example 1, which does not have a barrier layer, the barrier layer prevents the migration of glycerin. It was also recognized that the evaluation results of the migration of glycerin in Examples 1 to 3 were “A,” whereas the evaluation results of the migration of glycerin in Examples 4 to 6 were “B.” These results suggest that from the viewpoint of preventing the migration of glycerin, the material that forms the barrier layer is preferably a polyvinyl butyral resin.

It was also recognized that among Examples 2 and 4 to 6, the energy decrease rate was lowest in Example 2. These results suggest that from the viewpoint of maintaining the elasticity of the patch tape at a high level, the material that forms the barrier layer is preferably a polyvinyl butyral resin.

It can also be said that from the viewpoint of maintaining the elasticity of the patch tape at a high level, the energy decrease rate is preferably 70% or less, more preferably 50% or less, and even more preferably 40% or less.

According to the skin patch sheet described above, the effects described below can be obtained.
(1) Since the 180° peeling strength between the carrier sheet 11 and the substrate layer 21 is 550 mN/25 mm or less, the carrier sheet 11 can be easily peeled from the substrate layer 21. Further, the thickness of the barrier layer 23 being less than 5 μm prevents the deformation of the patch tape 13 when the patch tape 13 is stretched or compressed, and also prevents tearing of the patch tape 13 when the patch tape 13 is peeled from the skin.
(2) Since the decrease rate of the energy at the second 100% elongation is 70% or less, with the energy at the first 100% elongation as the reference value, even the skin patch sheet 10, which includes the barrier layer 23, can maintain elasticity.
(3) The barrier layer 23 prevents the active ingredient from migrating from the adhesive layer 22 toward the substrate layer 21, thereby preventing the increase in the adhesion between the carrier sheet 11 and the substrate layer 21.
(4) Since the barrier layer 23 is made of a synthetic resin, the increase in the rigidity of the barrier layer 23 can be prevented.

What is claimed is:

1. A skin patch sheet comprising:
a carrier sheet;
a release sheet; and
a patch tape located between the carrier sheet and the release sheet,
the patch tape comprising:
a substrate layer in contact with the carrier sheet;
an adhesive layer containing an active ingredient for skin and an adhesive; and
a barrier layer located between the substrate layer and the adhesive layer and having a thickness of less than 5 μm,
the patch tape being attached to the skin after the release sheet is peeled from the adhesive layer,
a 180° peeling strength according to JIS Z 0237:2009 between the carrier sheet and the substrate layer being 550 mN/25 mm or less, and
in the patch tape, a tensile elongation at break according to JIS K 7161-1:2014 being 130% or more.

2. The skin patch sheet of claim 1, wherein
when the patch tape is subjected to 100% elongation according to JIS K 7161-1:2014 twice in a row, the decrease rate of energy at the second 100% elongation is 70% or less, with energy at the first 100% elongation as a reference value, and
the energy is an integral value of test force and displacement obtained by the 100% elongation.

3. The skin patch sheet of claim 1, wherein
the active ingredient is hydrophilic,
the substrate layer includes a first surface in contact with the barrier layer and a second surface on an opposite side to the first surface, and
regarding a contact angle according to the sessile drop method of JIS R 3257:1999, the decrease rate of the contact angle 20 seconds after a drop of water is dropped is 25% or less, with the contact angle when the drop of water is dropped on the second surface as a reference value.

4. The skin patch sheet of claim 1, wherein the barrier layer is made of a synthetic resin.

5. The skin patch sheet of claim 1, wherein
the substrate layer contains a polyurethane resin, and
the adhesive layer contains a polyurethane resin.

6. The skin patch sheet of claim 1, wherein a surface of the carrier sheet in contact with the substrate layer is made of a polyolefin resin.
